(19) United States
(12) Patent Application Publication
(10) Pub. No.: US 2013/0312760 A1
(43) Pub. Date: Nov. 28, 2013

(54) Soft Touch Nose & Ear Pads

(71) Applicants: Madelon Kostyk, Huntington Beach, CA (US); Nadine Mariotti, Fountain Valley, CA (US)

(72) Inventors: Madelon Kostyk, Huntington Beach, CA (US); Nadine Mariotti, Fountain Valley, CA (US)

(21) Appl. No.: 13/479,845

(22) Filed: May 24, 2012

(51) Int. Cl. A61M 16/00

(52) U.S. Cl. 128/207.18

(57) ABSTRACT

The invention provides a soft covering for the plastic tubing on the cannula loop thereby . reducing friction and irritation to the patient's ears and cheeks. The pads are soft, neoprene cylinders which fit loosely around the plastic tubing of the cannula loop. The cannula loop can continue to move within the neoprene padded cylinders, while the cylinders themselves remain stationary at the contact points along the bony prominences of the cheek and behind the ears of the patient. Friction and chafing are reduced because the soft touch cylinder pads protect the skin contact points while allowing the cannula loop to move as the patient moves.

### CROSS REFERENCE TO RELATED APPLICATIONS

There are no related applications.

### STATEMENT REGARDING FEDERALLY SPONSORED RESEARCH & DEVELOPMENT

This invention was not made by an agency of the United States Government or under a contract with an agency of the United States Government. This invention was not made under Federally-Sponsored Research and Development. The inventor(s) retain all rights.

### NAMES OF THE PARTIES TO JOINT RESEARCH AGREEMENT

N/A

### REFERENCE TO SEQUENCE LISTING

N/A

### BACKGROUND OF THE INVENTION

A nasal cannula is a narrow, flexible plastic tubing used to deliver oxygen to patients through their nostrils. A loop with dual-pronged, extended openings is connected to one end of the cannula plastic tubing. The other end of the tubing is connected to an oxygen outlet, tank source or compressor. The cannula loop prongs are slightly curved to fit readily into the front portion of a patient's nostrils. Once the prongs are placed in the nostrils, the tubing of the loop is positioned along the patient's cheeks, and fitted over the patient's ears. The remaining loop tubing is brought together under the patient's chin by sliding a connector that holds the cannula loop in place.

The purpose of nasal cannula loops is to deliver oxygen in as concentrated a form as required for patients who are hypoxic. There are many conditions that cause hypoxemia and require the administration of supplemental oxygen, including respiratory disease, cardiac disease, shock, trauma, severe electrolyte imbalance (hypokalemia), low hemoglobin or severe blood loss, and seizures. Prompt treatment of these conditions with non-invasive oxygen administration can prevent the need for more invasive procedures.

Patients using nasal cannula loops on a regular basis, often suffer from skin chafing along the cheeks and behind the ears where the plastic tubing makes contact. This is where skin breakdown can happen the avoidance of which is a primary objective of this invention. Other product manufacturers have attempted to resolve the chafing issue using materials such as Styrofoam, but that material can be just as abrasive to the skin as the plastic tubing itself. The present invention addresses the chafing problem differently.

### FIELD OF THE INVENTION

The invention is basically an accessory to a commonly used medical device used by patients requiring concentrated oxygen delivery through the nostrils. Its purpose is to make the experience of wearing a nasal cannula loop more comfortable for those patients and to prevent skin breakdown and pressure sores. Medicare doesn't pay for skin breakdown treatment that occurs in the hospital when the patient did not have skin breakdown upon admission. Prevention of skin breakdown is therefore a desirable objective for hospitals.

### DESCRIPTION OF RELATED ART

As mentioned above, other product manufacturers have attempted to resolve the chafing issue associated with long-term use of nasal cannula loops using materials such as Styrofoam, or other compounds.

### BRIEF SUMMARY OF THE INVENTION

The invention provides a soft covering for the plastic tubing on the cannula loop thereby reducing friction and irritation to the patient's ears and cheeks. The pads are soft, neoprene cylinders which fit loosely around the plastic tubing of the cannula loop. The cannula loop can continue to move within the neoprene padded cylinders, while the cylinders themselves remain stationary at the contact points along the chin and behind the ears of the patient. Friction and chafing are reduced because the soft touch cylinder pads protect the skin contact points while allowing the cannula loop to move as the patient moves. The cannula covering in this invention can be individualized to each patient's face shape and size such as by varying the length and position of the pads. The pads are in one piece in the sense that they cannot be removed from around the outside of the tube but they can be slid along the tube in either direction to customize their placement for each individual patient.

### BRIEF DESCRIPTION OF THE DRAWINGS

FIG. 1: Left side view of cannula loop with soft touch pads Positioned along the left cheek and left ear contact points.

FIG. 2 Front view of cannula loop fitted into nostrils with soft Touch pads along both cheeks of the patient

### DETAILED DESCRIPTION OF INVENTION

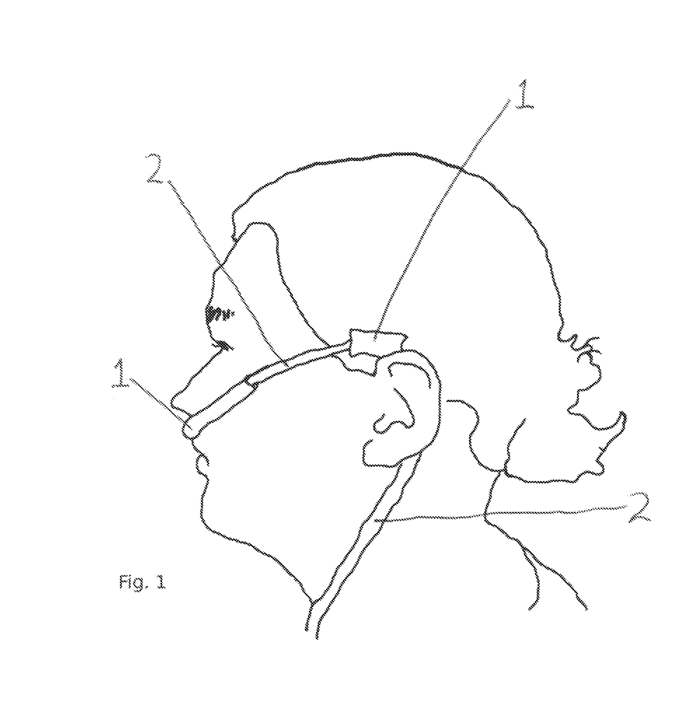

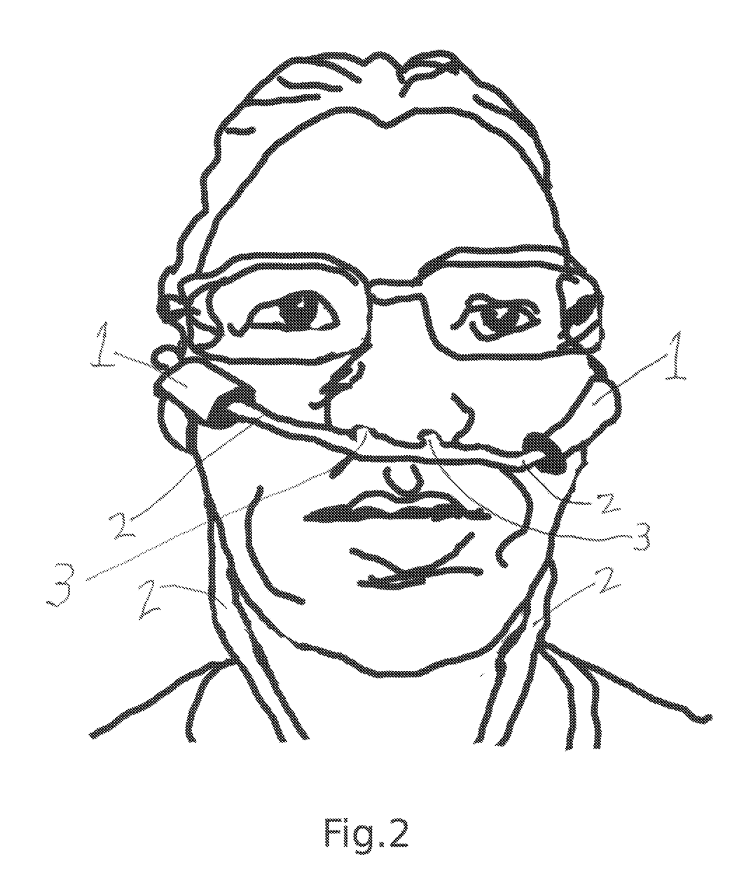

The invention provides a soft covering for the plastic tubing on the cannula loop thereby reducing friction and irritation to the patient's ears and cheeks. Referring now to FIG. 1 the pads (1) are soft, neoprene cylinders which fit loosely around the plastic tubing (2) of the cannula loop. The cannula loop can continue to move within the neoprene padded cylinders, while the cylinders themselves remain stationary at the contact points along the chin and behind the ears of the patient. Referring now to FIG. 2, the pads (1) can be seen to surround the cannula tube (2) so soft padding is against the patient's cheeks while the nasal plugs (3) are inserted for the delivery of oxygen. Friction and chafing are reduced because the soft touch cylinder pads protect the skin contact points while allowing the cannula loop to move as the patient moves. The cannula covering in this invention can be individualized to each patient's face shape and size such as by varying the length and position of the pads. The tubing and pads may be manufactured in colors or with patterns that are aesthetically pleasing in order to improve patient compliance with wearing of the nasal cannula. The pads are in one piece in the sense that they cannot be removed from around the outside of the tube but they can be slid along the tube in either direction to customize their placement for each individual patient. In a preferred embodiment the cannula would have several pads and the pads would be about 1.5 inches long and slidable so that if necessary two pads can be slid together to form three inches of padding on a patient with a larger area of skin contact with the cannula tubing.

The materials given from which the pads can be manufactured are merely illustrative and other equivalent soft materials may be used in keeping with the objectives of this invention. Similarly, the lengths of the pads mentioned are merely illustrative of one embodiment of the invention and other lengths may be used up to and including having the entire outside of the cannula tube covered in padding. The nasal prongs may be padded themselves in certain applications without departing from the spirit of this invention and the inventors do not intend to be limited to only the specific preferred embodiment described herein.

We claim:

1. A pad for a nasal cannula tube comprising a soft cylinder having a bore through its axis, said nasal cannula tube passing through said bore.

2. The pad for a nasal cannula tube of claim 1 in which said nasal cannula tube can slide freely through said bore.

3. The pad for a nasal cannula tube of claim 2 in which said soft cylinder is NEOPRENE brand synthetic foam material.

4. A nasal cannula tube comprising two hollow nasal prongs attached to a length of tubing in a substantially perpendicular manner and suitably spaced to fit human nostrils wherein said hollow nasal prongs permit exit of gas flowing through said length of tubing; said length of tubing having an exterior; and at least one soft cylinder having a bore through its axis surrounding said exterior.

5. The nasal cannula tube of claim 4 wherein at least one said soft cylinder having a bore through its axis surrounds said exterior of said length of tubing on each side of said nasal prongs.

6. The nasal cannula tube of claim 5 wherein said length of tubing can slide freely through said bores.

7. The nasal cannula tube of claim 6 having a plurality of said soft cylinders on each side of said hollow nasal prongs and wherein said soft cylinders are NEOPRENE brand foam material.

8. The nasal cannula tube of claim 4 wherein said length of tubing can slide freely through said bore.
